(19) United States
(12) Patent Application Publication
(10) Pub. No.: US 2025/0376814 A1
(43) Pub. Date: Dec. 11, 2025

(54) SOIL COMPACTOR

(71) Applicant: Hamm AG, Tirschenreuth (DE)

(72) Inventors: Axel MÜHLHAUSEN, Kassel (DE); Rainer KROCKAUER, Plössberg (DE); Marco KREGER, Tirschenreuth (DE); Klaus MEINDL, Bärnau/Hohenthan (DE)

(21) Appl. No.: 19/228,332

(22) Filed: Jun. 4, 2025

(30) Foreign Application Priority Data
Jun. 5, 2024 (DE) 102024115572.8

(51) Int. Cl. E01C 19/26; G01N 33/42; G01S 13/88

(52) U.S. Cl. CPC E01C 19/26; G01N 33/42; G01S 13/885

(57) ABSTRACT

A soil compactor comprises at least one compactor roller (18, 20) rotatably supported on a compactor frame (12) and at least one soil radar density measuring arrangement (34) arranged in an inner volume region (VI) of the compactor frame (12).

The present invention relates to a soil compactor using which soil material, in particular asphalt, can be compacted.

WO 2021/229146 A1 discloses a soil compactor designed as a so-called roller train, in which a compactor frame comprises a main frame with a drive unit provided thereon, a control station and drive wheels driven by the drive unit. A sub-frame is connected to the main frame so as to be pivotable about a substantially vertically oriented pivot axis. A compactor roller is rotatably supported on the sub-frame about a roller axis of rotation extending essentially in a soil compactor transverse direction and perpendicularly to a soil compactor longitudinal direction. A soil radar moisture measuring arrangement is supported on an underside of the main frame, projecting downwards in the vertical direction, in such a way that an antenna of this soil radar moisture measuring arrangement is positioned at a small distance from the soil to be detected with regard to its moisture content.

It is the object of the present invention to provide a soil compactor with a soil radar density measuring arrangement protected against external influences.

According to the invention, this object is achieved by a soil compactor comprising at least one compactor roller rotatably supported on a compactor frame and at least one soil radar density measuring arrangement arranged in an inner volume region of the compactor frame.

By arranging at least one soil radar density measuring arrangement in an inner volume region of the compactor frame, i.e. not projecting outwards on the compactor frame, such a soil radar density measuring arrangement is supported on the soil compactor essentially protected against physical action from the outside. Since obstacles which can collide with the soil compactor are frequently present in regions in which such soil compactors operate, damage to a soil radar density measuring arrangement accommodated in this way due to an obstacle colliding with the soil compactor can be avoided. Also during cleaning operations, for example by means of a high-pressure cleaner, there is no risk of damage to a soil radar density measuring arrangement accommodated on the soil compactor.

The compactor frame can comprise a main frame and at least one sub-frame which is connected to the main frame such that it can pivot about a steering axis, wherein a drive unit to be operated for driving the soil compactor is provided on the main frame and a compactor roller is rotatably supported on the at least one sub-frame.

Such a soil compactor can be designed as a so-called tandem roller with a compactor roller rotatably supported on the main frame and a compactor roller rotatably supported on an sub-frame, as a so-called roller train with a compactor roller rotatably supported on the sub-frame and drive wheels supported on the main frame, or can be designed as a so-called pivot-steered soil compactor, in which two sub-frames designed as steering frames are pivotably mounted on the main frame and a compactor roller is rotatably supported on each sub-frame.

In addition, in such a soil compactor, independently of its construction, one or more compactor rollers can be provided on the main frame and/or on at least one sub-frame as a compactor roller which is constructed as a continuous or divided compactor roller in the direction of a roller axis of rotation and which has a roller shell which is generally constructed of steel material. Alternatively or additionally, in the case of such a soil compactor, one or more compactor rollers can each be designed with a plurality of rubber wheel rollers arranged successively in the direction of a roller axis of rotation.

For the pivotable connection, the at least one sub-frame can comprise a cross member extending in a soil compactor transverse direction, the sub-frame being pivotably connected to the main frame in the region of the cross member.

At least one soil radar density measuring arrangement can be arranged in a sub-frame inner volume region of the cross member. For this purpose, the cross member can be designed as a hollow support or hollow profile support for providing a sub-frame inner volume region.

In particular, when the soil compactor is designed as a tandem roller or roller train, the at least one sub-frame can comprise a further cross member which is arranged at a distance from the cross member in a longitudinal direction of the soil compactor and which is connected to the cross member by two further longitudinal members which are arranged at a distance from one another in the transverse direction of the soil compactor and extend substantially in the longitudinal direction of the soil compactor and which further cross member extends substantially in the transverse direction of the soil compactor, the compactor roller being rotatably supported on the longitudinal members. At least one soil radar density measuring arrangement can be arranged in a sub-frame inner volume region of the further cross member and/or in a sub-frame inner volume region of at least one longitudinal member.

For this purpose, it can be provided that the further cross member and/or at least one of the longitudinal members is configured for providing a sub-frame inner volume region as a hollow support or hollow profile support.

If the soil compactor comprises two sub-frames designed as steering frames, which are connected to the main frame so as to be pivotable about a respective steering axis, at least one soil radar density measuring arrangement can be arranged in a sub-frame inner volume region of at least one of the sub-frames designed as steering frames.

In a further embodiment of the soil compactor according to the invention, the main frame can have a main frame inner volume region containing the drive unit and at least one soil radar density measuring arrangement.

In order to carry out maintenance work on the drive unit or in connection with the additional units carried on the main frame, at least one preferably closable inspection opening can be provided in the main frame. In this case, at least one soil radar density measuring arrangement can be positioned in such a way that it, preferably each soil radar density measuring arrangement arranged in the main frame inner volume region, is accessible via at least one inspection opening. This means that a soil radar density measuring arrangement positioned in this way can be maintained through the inspection opening and, if necessary, replaced.

In order to ensure that one or more soil radar density measuring arrangements are accommodated in the compactor frame of the soil compactor in such a way that they are protected against vibrations, it is proposed that at least one, preferably each soil radar density measuring arrangement comprises a sensor carrier which is carried on the compactor frame by means of at least one elastically deformable suspension element, and at least one soil radar density sensor which is carried on the sensor carrier.

For a stable mounting on the compactor frame, at least one, preferably each, soil radar density measuring arrangement can be carried on a plate-like carrier region of the compactor frame.

The detection of the soil lying under or in the region of a soil compactor by means of a soil radar density measuring arrangement arranged in an inner volume region of the soil compactor can be made possible in that a measuring opening is provided on the compactor frame in association with at least one, preferably each, soil radar density measuring arrangement.

In order to be able to comprehensively detect a compaction state of a soil to be compacted generated by the soil compactor, it is proposed that, in association with at least one, preferably each, compactor roller, at least one soil radar density measuring arrangement is provided on each side of the compactor roller in a longitudinal direction of the soil compactor, and/or that at least two soil radar density measuring arrangements are arranged at a distance from one another in a transverse direction of the soil compactor.

The present invention is described in detail below with reference to the attached figures. In particular:

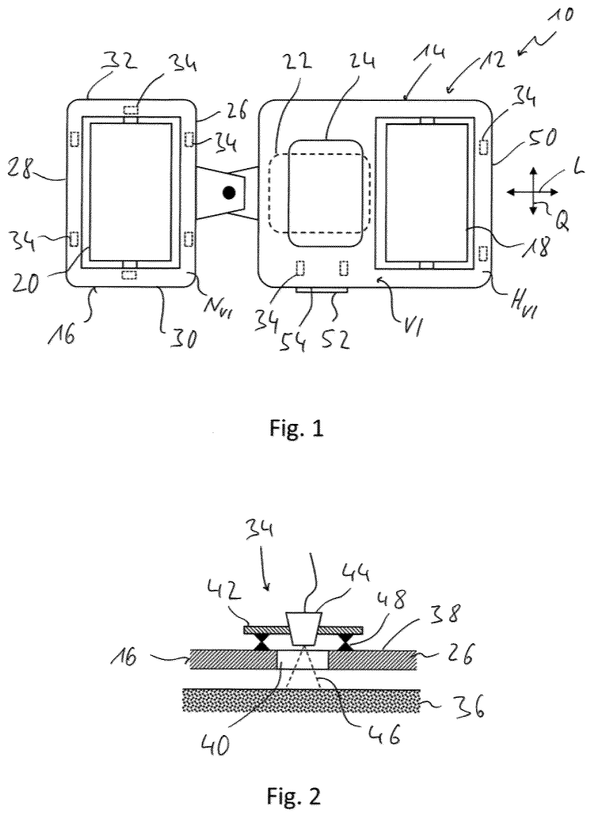

FIG. 1 shows in principle a plan view of a soil compactor designed as a tandem roller;

FIG. 2 shows in principle a soil radar density measuring arrangement arranged in an inner volume region of the soil compactor;

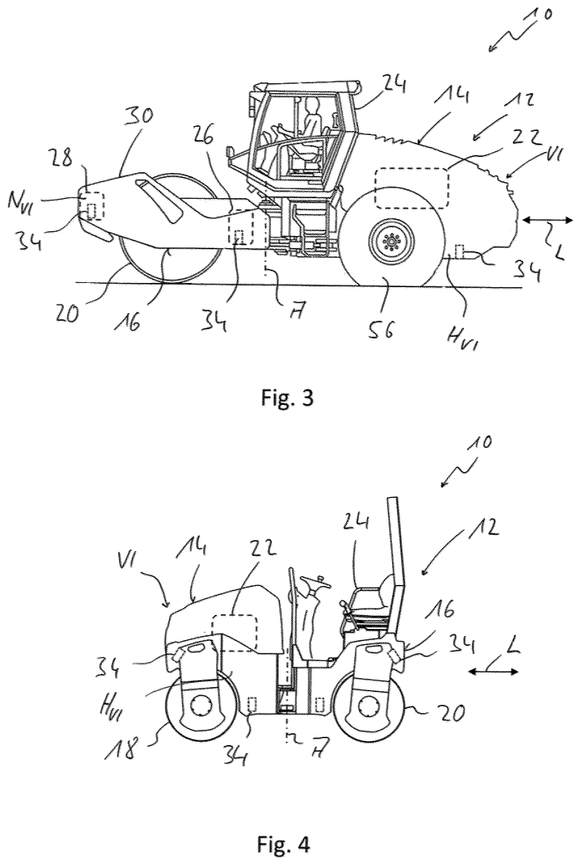

FIG. 3 is a side view of a soil compactor designed as a roller train;

FIG. 4 is a side view of a small roller constructed as a tandem roller;

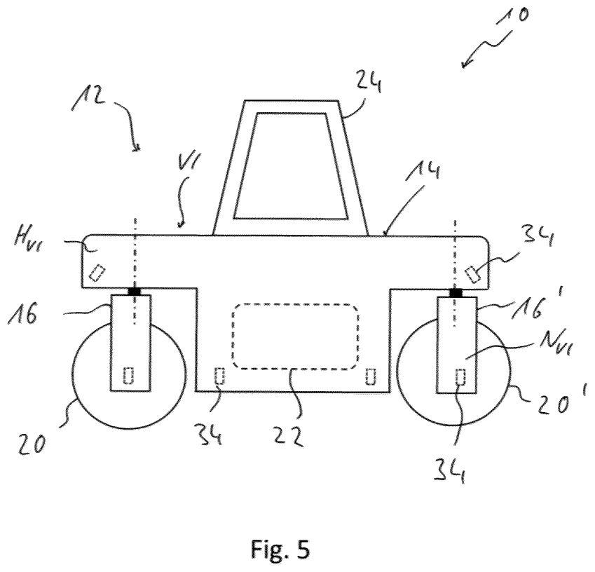

FIG. 5 is a principle side view of a sub-frame-steered soil compactor.

The soil compactor 10 shown in FIG. 1 comprises a compactor frame generally designated 12 with a main frame 14 and a sub-frame 16 pivotably connected to the main frame 14 about a steering axis A which is substantially orthogonal to the plane of the drawing in FIG. 1. A compactor roller 18 is rotatably supported on the main frame 14 about a roller axis of rotation extending in a soil compactor transverse direction Q and substantially orthogonal to a soil compactor longitudinal direction L. A compactor roller 20 is rotatably supported on the sub-frame 16 about a roller axis of rotation extending essentially in a soil compactor transverse direction Q and perpendicularly to a soil compactor longitudinal direction L.

A drive unit 22, which is designed, for example, with an internal combustion engine or an electric motor, is supported on the main frame 14 and provides the drive energy for operating the soil compactor 10. In addition, an operating station 24 for an operator operating the soil compactor 10 is provided on the main frame 14.

The sub-frame 16 comprises a cross member 26 extending substantially in the transverse direction Q of the soil compactor, by means of which the sub-frame 16 is pivotably connected to the main frame 14. A further cross member 28 is arranged in the longitudinal direction L of the soil compactor at a distance from the cross member 26 and extending substantially parallel thereto and is connected to the cross member 26 by two longitudinal members 30, 32 extending substantially in the longitudinal direction L of the soil compactor and arranged at a distance from one another in the transverse direction Q of the soil compactor, so that the sub-frame 16 substantially completely surrounds the compactor roller 20, which is rotatably supported on the longitudinal members 30, 32 thereof. At least one of the two cross members 26, 28 and/or at least one of the longitudinal members 30, 32 is designed as a hollow member and thus provides a sub-frame inner volume region N_{VI} in which at least one soil radar density measuring arrangement 34 is arranged. By means of such a soil radar density measuring arrangement 34, information representing the compaction state or density of the soil or subsoil over which the soil compactor 10 passes can be generated by scanning and made available for further evaluation.

A soil radar density measuring arrangement 34 of this type, which is arranged, for example, on the cross member 26 of the sub-frame 16, is illustrated in FIG. 2. In association with this soil radar density measuring arrangement 34, a measuring opening 40 is formed on a plate-like region 38 of the cross member 26, which is designed as a hollow member and faces the soil 36 to be compacted or already compacted, through which measuring opening there is access for detection purposes to the section of the soil 36 lying below this region of the sub-frame 16. The soil radar density measuring arrangement 34 comprises a plate-like sensor carrier 42, for example, on which a soil radar density sensor 44 is supported in the illustrated embodiment in such a way that its measurement observation field 46 is directed downwards through the measurement opening 40 onto the soil 36. In order to protect the soil radar density sensor 44 against vibrations or other vibrations, the sensor carrier 42 is supported on the transverse carrier 26 or the plate-like region 38 thereof by means of a plurality of elastic suspension elements 48 constructed, for example, with rubber material.

In order to be able to detect in particular the change in the compaction state of the soil 16 generated by the compactor roller 20, it is advantageous to provide at least one such soil radar density measuring arrangement 34 on each side of the compactor roller 20 in the longitudinal direction L of the soil compactor. This means that a soil radar density measuring arrangement 34 of this type can also be provided in the further cross member 28 which is located at a greater distance from the main frame 14, for example in the manner shown in FIG. 2.

In order to be able to provide information about the state of compaction in the width direction, i.e. the transverse direction Q of the compactor, of the soil being passed over, it is further advantageous to provide soil radar density measuring arrangements 34, which are spaced apart from one another in the transverse direction Q of the compactor, for example on the cross member 26 and/or on the further cross member 28.

As an alternative or in addition to providing one or more soil radar density measuring arrangements 34 in one or in both cross members 26, 28, in particular when the longitudinal members 30, 32 are designed as hollow members, one or more density measuring arrangements can also be provided therein.

Alternatively or in addition to providing one or more soil radar density measuring arrangements 34 on the sub-frame 16, one or more such soil radar density measuring arrangements 34 can also be provided on the main frame 14. For example, it is possible to accommodate one or more soil radar density measuring arrangements 34 in a cross member 50 of the main frame 40. It is particularly advantageous to provide such soil radar measuring arrangements 34 in the region of an inspection opening 54 of the main frame 14 which can be closed by a plate-like closure element 52 and through which access is provided to the drive unit 22 or other units accommodated in a main frame inner volume region H_{VI}. For example, such soil radar density measuring arrangements 34 provided in the region of the inspection opening 54 can be positioned on a base plate of the main frame 14 opposite the soil to be compacted or already compacted in a manner corresponding to FIG. 2.

By accommodating one or more soil radar density measuring arrangements 34 in an inner volume region VI of the soil compactor 10, that is to say a sub-frame inner volume region N_{VI} and/or a main frame inner volume region H_{VI}, an accommodation which is protected against external influences is ensured, which nevertheless ensures reliable, exact detection of the state of the soil, in particular on account of the positioning of such soil radar density measuring arrangements 34 shown in FIG. 2.

FIG. 3 shows a soil compactor 10 designed as a roller train with a main frame 14, generally also referred to as a rear carriage, and a sub-frame 16, generally referred to as a front carriage. These are connected to one another pivotably about a steering axis A. The drive unit 22 and the operating station 24 are accommodated on the main frame 14. Furthermore, two drive wheels 56 are provided on the main frame 14, which can be driven by the drive unit 22 for rotation and thus also for advancing the soil compactor 10.

The sub-frame has the two cross members 26, 28 and the longitudinal members 30, 32 connecting the latter, which longitudinal members surround the compactor roller 20 rotatably supported on the sub-frame 16.

As illustrated in FIG. 3, for example, one or more soil radar density measuring arrangements 34 can be accommodated in one or both of the cross members 26, 28 in the manner described above with reference to FIG. 2. If the longitudinal members 30, 32 are designed as hollow members, one or more soil radar density measuring arrangements 34 can alternatively or additionally also be accommodated therein. One or more soil radar density measuring arrangements 34 can also be arranged on the main frame 14, for example in a rear region thereof, or soil radar density measuring arrangements 34 can be provided which are positioned at a distance from one another in the longitudinal direction L of the soil compactor.

An alternative embodiment of a soil compactor 10 is shown in FIG. 4. The soil compactor 10 of FIG. 4 is basically designed as a tandem roller with two compactor rollers 18, 20. The compactor roller 20 is supported on a main frame 14 which also supports the drive unit 32. The compactor roller 20 is supported on the sub-frame 16 having the operating station 24. The main frame 14 and the sub-frame 16 are supported so as to be pivotable relative to one another about the steering axis A. One or more soil radar density measuring arrangements 34 can be carried on the main frame 14 and/or on the sub-frame 16, for example on a respective base plate in the manner shown in FIG. 2. It is also possible to position, for example on the main frame 14, one or more soil radar density measuring arrangements 34 in the main frame inner volume region H_{VI} in such a way that these are directed obliquely forwards onto the soil positioned in front of the compactor roller 18 of the main frame 14, in order to detect, depending on the direction of movement, the state of compaction before or after the soil has been traveled over by the soil compactor 10. A corresponding positioning is also possible on the sub-frame 16.

FIG. 5 shows a pivot-steered soil compactor 10 in which two sub-frames 16, 16′, which are designed as steering frames, are supported on the main frame 14 so as to be pivotable about respective steering axes A, A′. A compactor roller 20, 20′ is rotatably supported on each of the sub-frames 16, 16′ designed as steering frames. One or more soil radar density measuring arrangements 34 can be accommodated both in a main-frame inner-volume region H_{VI} and in respective sub-frame inner-volume regions N_{VI} of the sub-frames 16, 16′ in such a way that they can detect the soil lying under the soil compactor 10 through respective measuring openings and thus provide information about its compaction state or density.

Finally, it should be pointed out that if several soil radar density measuring arrangements 34 are provided on the above-described soil compactor 10, these may be accommodated at any of the above-described positions shown in the figures. Particularly advantageous is an arrangement in which at least two soil radar density measuring arrangements 34 are arranged at a distance from one another in the longitudinal direction L of the soil compactor, for example in such a way that they are arranged on both sides of a compactor roller 18 or 20 in the longitudinal direction L of the soil compactor, and/or at least two soil radar density measuring arrangements 34 are arranged at a distance from one another in the transverse direction Q of the compactor. For example, if two soil radar density measuring arrangements 34 are used, one of these soil radar density measuring arrangements 34 could be arranged in the cross member 26 in the region of one longitudinal end in the transverse direction of the soil compactor, while the other soil radar density measuring arrangement 34 is arranged in the further cross member 28 in the region of the other longitudinal end, that is to say in the transverse direction Q of the soil compactor on or near the other side of the soil compactor 10.

1. A soil compactor comprising at least one compactor roller rotatably supported on a compactor frame and at least one soil radar density measuring arrangement arranged in an inner volume region of the compactor frame.

2. The soil compactor according to claim 1,
wherein the compactor frame comprises a main frame and at least one sub-frame pivotably connected to the main frame about a steering axis, wherein a drive unit to be operated for driving the soil compactor is provided on the main frame and a compactor roller is rotatably supported on the at least one sub-frame.

3. The soil compactor according to claim 2,
wherein the at least one sub-frame can comprise a cross member extending in a soil compactor transverse direction, the sub-frame being pivotably connected to the main frame in the region of the cross member.

4. The soil compactor according to claim 3,
wherein at least one soil radar density measuring arrangement is arranged in a sub-frame inner volume region of the cross member.

5. The soil compactor according to claim 4,
wherein the cross member is designed as a hollow member for providing a sub-frame inner volume region.

6. The soil compactor according to claim 3, wherein the at least one sub-frame comprises a further cross member arranged in a longitudinal direction of the soil compactor at a distance from the cross member and connected to the cross member by two longitudinal members arranged at a distance from one another in the transverse direction of the soil compactor and extending substantially in the longitudinal direction of the soil compactor, and which extends substantially in the soil compactor transverse direction, wherein the compactor roller is rotatably supported on the longitudinal members, and in that at least one soil radar density measuring arrangement is arranged in a sub-frame inner volume region of the further cross member or/and in a sub-frame inner volume region of at least one longitudinal member.

7. The soil compactor according to claim 6,
wherein the further cross member and/or at least one of the longitudinal members is designed as a hollow member for providing a sub-frame inner volume region.

8. The soil compactor according to claim 3,
wherein two sub-frames designed as steering frames are pivotably connected to the main frame about a respective steering axis, and in that at least one soil radar density measuring arrangement is arranged in a sub-frame inner volume region of at least one of the sub-frames designed as steering pivots.

9. The soil compactor according to claim 2, wherein the main frame has a main frame inner volume region containing the drive unit and at least one soil radar density measuring arrangement.

10. The soil compactor according to claim 9,
wherein at least one preferably closable inspection opening is provided in the main frame, and in that at least one, preferably each, soil radar density measuring arrangement arranged in the main frame inner volume region is accessible via at least one inspection opening.

11. The soil compactor according to claim 1, wherein at least one, preferably each soil radar density measuring arrangement comprises a sensor carrier carried on the compactor frame by means of at least one elastically deformable suspension element and at least one soil radar density sensor carried on the sensor carrier.

12. The soil compactor according to claim 1, wherein at least one, preferably each, soil radar density measuring arrangement is supported on a plate-like carrier region of the compactor frame.

13. The soil compactor according to claim 1, wherein a measuring opening is provided on the compactor frame in association with at least one, preferably each, soil radar density measuring arrangement.

14. The soil compactor according to claim 1, wherein at least one soil radar density measuring arrangement is provided on each side of the compactor roller in a longitudinal soil compactor longitudinal direction in association with at least one, preferably each, compactor roller, or/and in that at least two soil radar density measuring arrangements are arranged at a distance from one another in a soil compactor transverse direction.
